(19) United States
(12) Patent Application Publication
(10) Pub. No.: US 2016/0263152 A1
(43) Pub. Date: Sep. 15, 2016

(54) BOWEL CLEANSING COMPOSITION WITH IMPROVED TASTE

(71) Applicant: Gavis Pharmaceuticals, Somerset, NJ (US)

(72) Inventors: Aman Trehan, Somerset, NJ (US); James Garegnani, Somerset, NJ (US)

(73) Assignee: Gavis Pharmaceuticals, Somerset, NJ (US)

(21) Appl. No.: 15/055,930

(22) Filed: Feb. 29, 2016

Related U.S. Application Data
(60) Provisional application No. 62/133,332, filed on Mar. 14, 2015.

(51) Int. Cl. A61K 33/08; A61K 31/194; A61K 31/4402

(52) U.S. Cl. CPC A61K 33/08; A61K 31/4402; A61K 31/194; A23L 1/22091; A23V 2002/00

(57) ABSTRACT

The present invention provides compositions and methods for facilitating cleansing of the gastrointestinal tract of a patient prior to a diagnostic, surgical or therapeutic procedure. The composition can improve patient compliance, and thus, efficacy of the preparation. Specifically, the composition of the invention is palatable for the patient to consume. For example, for a patient preparing to undergo colonoscopy, the present methods make the bowel preparation liquid taste significantly less salty.

### CROSS-REFERENCE TO RELATED APPLICATIONS

This application is a nonprovisional application claiming the benefit priority of U.S. Provisional Application No. 62/133,332, filed Mar. 14, 2015, the contents of which are incorporated herein in their entirety by reference.

### BACKGROUND OF THE INVENTION

(a) Field of the Invention

The present invention relates to a pharmaceutical composition for cleansing the gastrointestinal tract of a patient prior to a diagnostic, surgical or therapeutic procedure. In particular, this invention makes the composition palatable.

(b) Description of the Related Art

Colorectal cancer is the third most common cancer among both men and women in the world. Early detection of colorectal cancer greatly improves the chances of a cure. Colonoscopies are widely recognized as the gold standard for colorectal cancer screening (Rex et al., Colorectal cancer prevention 2000: Screening recommendations of the American College of Gastroenterology. Am. J. Gastroenterol. 2000; 95: 868-877). Colonoscopies are also frequently used to diagnose many other gastrointestinal pathologies.

Despite the effectiveness of colonoscopies, compliance is often an issue among patients, mainly due to the bowel preparation procedure where the patient is required to drink large volumes of a foul-tasting, or at least an unpleasant tasting, solution. For the colonoscopy test to be performed properly, the colon must be free of solid matter. Thus, prior to undergoing a colonoscopy, the patient needs to ingest bowel preparation solutions to empty the bowel.

The preparations typically contain large amounts of polyethylene glycol and electrolytes (e.g., sodium chloride, sodium bicarbonate, and/or potassium chloride). A large amount (e.g., 4 litres) of this salty, foul-tasting solution must be taken orally to cleanse the bowel. The bowel preparation procedure is often described as very unpleasant by colonoscopy recipients (U.S. Patent Publication No. 20090053304).

Inadequate preparations are responsible for up to one third (⅓) of all incomplete procedures, preclude up to 10% of examinations, and negatively impact the rate of polyp and adenoma detection (Technology Status Evaluation Report: Colonoscopy Preparation, Gastrointestinal Endoscopy, 2009, 69(7):1201-1209). Because of the importance of proper cleansing of the colon for success of the colonoscopy, there has been an increased focus on the palatability of the solution as a factor of patient compliance (The Prep Is Worse Than The Procedure, Harvard Health Newsletters, Jan 1, 2010).

The ideal bowel preparation is safe, effective and acceptable to patients with negligible discomfort. Because it is safer and more effective than other prep solutions, the polyethylene glycol (PEG) solution has been used as the so-called “gold standard” for colonoscopy. However, despite the fact that PEG solutions are well tolerated by patients, 5%-15% of patients do not complete the preparation because of poor palatability and/or the large volume required to be ingested (R H Hawes et al., Consensus Document on Bowel Preparation before Colonoscopy, Gastrointestinal Endoscopy, 2006, 63(7): 894-909).

Efforts have been made to make bowel preparation solutions more palatable with the addition of flavourings. For example, polyethylene glycol solutions are available in multiple flavours, such as cherry, citrus-berry, lemon-lime, orange and pineapple. Sulfate salts have been removed from gastrointestinal tract preparation solutions such as HalfLytely® and NuLYTELY® resulting in a less salty taste and a less pungent “rotten egg” smell. Water, ginger ale, Gatorade, CrystalLite, and carbohydrate-electrolyte solutions have also been used to improve the taste of these solutions. However, flavouring packages do not significantly change palatability in terms of the saltiness and overall taste. Furthermore, improved flavour does not necessarily equate to improved tolerance. In fact, when flavouring additions are added, special care must be taken to avoid altering the osmolarity of the preparation or adding substrates to the preparation which can metabolize into explosive gases or alter the amount of water and salts absorbed (R H Hawes et al., Consensus Document on Bowel Preparation before Colonoscopy, Gastrointestinal Endoscopy, 2006, 63(7): 894-909).

U.S. Patent Publication Nos. 20130149390 and 20140037775 teach a method for cleansing the gastrointestinal tract and improving palatability using a taste-modifying substance. The method requires determining the pH of a cleansing composition having a salty taste, and adjusting the pH to a range of from about 3 to about 6.4, then administering the taste-modifying substance to the patient, followed by administering said pH adjusted preparation to the patient.

U.S. Pat. No. 7,169,381 teaches a composition effective in cleansing the gut in preparation for an endoscopy, especially colonoscopy, comprising, per litre of aqueous solution, from 30 to 350 grams of polyethylene glycol, from 3 to 20 gram of an ascorbic acid component selected from the group consisting of ascorbic acid, a salt of ascorbic acid, or a mixture thereof, an alkali metal or alkaline earth metal sulphate, from 1 to 15 grams thereof, and preferably also comprising flavourings. The patent teaches that a cleansing solution comprising an alkali metal or alkaline earth metal sulphate, ascorbic acid and/or one or more salts thereof, a relatively high concentration of polyethylene glycol has a cleansing action that is effective when administered in a small volume, and is palatable.

Despite the known methods and formulations proposed in the prior art for preparations for cleansing the gastrointestinal tract aiming to improve palatability, there is still a need for development of alternative and palatable bowel preparation compositions that would achieve effective cleansing with improved tolerability and reduced adverse effects. Such compositions additionally also should provide a simple method of cleansing the gastrointestinal tract.

### SUMMARY OF THE INVENTION

The present invention provides a pharmaceutical composition for cleansing the gastrointestinal tract of a subject. The composition includes one or more excipients and the pH of the composition is adjusted in the range of from about 3.0 to about 4.0.

In one aspect, the present invention provides a pharmaceutical composition for cleansing the gastrointestinal tract of a subject, the composition having a pH adjusted in the range of from about 3.0 to about 4.0 and the composition is at least about 30%, at least about 50%, or at least about 70% more palatable than the composition in which pH is not adjusted in the range from about 3.0 to about 4.0, wherein said composition is devoid of ascorbic acid, a salt of ascorbic acid, or a mixture thereof.

In another aspect, the invention provides a solid pharmaceutical composition for cleansing the gastrointestinal tract of a subject, wherein reconstitution of the composition in water results in a liquid that exhibits a pH in the range of from about 3.0 to about 4.0.

In a further aspect, the invention provides a method for cleansing the gastrointestinal tract of a subject comprising the steps of:
(a) determining the pH of a pharmaceutical composition for cleansing the gastrointestinal tract which has a salty taste and comprises at least one excipient,
(b) adjusting the pH of the composition in the range from about 3.0 to about 4.0; and
(c) orally administering the composition to the subject,
   wherein,
(i) said organic acid is not ascorbic acid, a salt of ascorbic acid, or a mixture thereof; and
(ii) the salty taste of the composition is reduced by at least about 30%, at least about 50%, or at least about 70%, compared to the salty taste of the composition had the pH not been adjusted in the range from about 3.0 to about 4.0.

In a further aspect, the invention provides a method of reducing the salty taste of a pharmaceutical composition for cleansing the gastrointestinal tract comprising adjusting the pH of said composition in the range from about 3.0 to about 4.0, wherein the salty taste of the composition is reduced by at least about 30%, at least about 50%, or at least about 70% compared to the salty taste of the composition had the pH not been adjusted in the range of from about 3.0 to about 4.0.

The composition is devoid of taste-modifying substances, or alternatively, may comprise one or more taste-modifying substances. Preferably, the composition is devoid of taste-modifying substances.

In a further aspect, the invention provides a pharmaceutical composition for cleansing the gastrointestinal tract of a subject, wherein the composition further may comprise polyethylene glycol.

In a further aspect, the invention provides a pharmaceutical composition for cleansing the gastrointestinal tract of a subject, wherein the composition comprises polyethylene glycol in an amount of about 20 gm to about 350 gm per litre of the composition.

In a further aspect, the invention provides a pharmaceutical composition for cleansing the gastrointestinal tract of a subject, wherein the composition comprises polyethylene glycol in an amount of less than 80 gm per litre of the composition.

In a further aspect, the invention provides a pharmaceutical composition for cleansing the gastrointestinal tract of a subject, wherein the composition further may comprise one or more alkali or alkaline earth metal sulfate salts, for example, sodium picosulfate.

In a further aspect, the invention provides a pharmaceutical composition for cleansing the gastrointestinal tract of a subject, wherein the composition further may comprise one or more sugars.

In a further aspect, the invention provides a pharmaceutical composition for cleansing the gastrointestinal tract of a subject comprising one or more sugars in amounts sufficient to improve taste but insufficient to produce intestinal gases upon administration.

In a further aspect, the invention provides a composition for cleansing the gastrointestinal tract of a subject, wherein the composition comprises sugar in an amount of less than 1.5 gm per litre of the composition.

In a further aspect, the gastrointestinal tract is the intestine and the composition is a bowel preparation solution.

In a further aspect, the invention provides a pharmaceutical composition for cleansing the gastrointestinal tract of a subject consisting essentially of:
(a) polyethylene glycol,
(b) an alkali or alkaline earth metal sulfate,
(c) at least one inorganic acid and/or organic acid; and
(d) optionally one or more electrolytes selected from sodium chloride, potassium chloride, sodium hydrogen carbonate, potassium hydrogen carbonate, and magnesium oxide,
   wherein—
(i) said organic acid is not ascorbic acid, a salt of ascorbic acid, or a mixture thereof,
(ii) the pH of said composition is adjusted in the range of from about 3.0 to about 4.0; and
(iii) said composition is at least about 30%, at least about 50%, or at least about 70% more palatable than the composition in which pH is not adjusted in the range from about 3.0 to about 4.0.

The pH of the composition may be adjusted by at least an inorganic acid or an organic acid. For example, the acids include, but are not limited to, hydrochloric acid, citric acid, acetic acid, phosphoric acid, malic acid, succinic acid, formic acid, fumaric acid, maleic acid, or mixtures thereof. The pH of the composition may also be adjusted by ammonium hydroxide, sodium carbonate, potassium carbonate, sodium bicarbonate, carbon dioxide, or mixtures thereof. Preferably, the organic acid used in the composition is other than ascorbic acid, a salt of ascorbic acid, or a mixture thereof.

The composition may contain a potassium salt, a sodium salt, a calcium salt, an ammonium salt or mixtures thereof. For example, the composition comprises sodium chloride, potassium chloride, and sodium bicarbonate (sodium hydrogen carbonate), or mixtures thereof. The composition may include at least one sodium phosphate. The composition may contain at least one alkali metal (e.g., sodium and potassium) salt, and/or at least one alkaline earth metal (e.g., magnesium or calcium) salt.

The composition may be a liquid, preferably in the form of a solution. The volume of the liquid composition can range from about 0.1 litres to about 5 litres or from about 1 litre to about 4 litres. Preferably, the volume of the liquid composition is 1 litre or less.

In another aspect, the present invention provides a method of cleansing the gastrointestinal tract prior to an endoscopy including colonoscopy and sigmoidoscopy, a barium enema examination, capsule endoscopy, colon surgery or gastrointestinal tract surgery, wherein the method comprises orally administering the pharmaceutical composition as substantially described herein.

The gastrointestinal tract may be cleansed prior to carrying out a diagnostic, therapeutic and/or surgical procedure on the patient. For example, the gastrointestinal tract is cleansed prior to an endoscopy, such as a colonoscopy or sigmoidoscopy. The gastrointestinal tract may be cleansed prior to a barium enema examination, capsule endoscopy, colon surgery or gastrointestinal tract surgery.

Still other aspects and advantages of the invention will be apparent from the following detailed description of the invention.

### DETAILED DESCRIPTION OF THE INVENTION

The present invention provides a composition and methods for cleansing the gastrointestinal tract of a patient using said composition prior to a diagnostic, surgical or therapeutic procedure. The methods of using the composition of the invention can improve patient compliance, and thus, efficacy of the preparation. Specifically, the present composition is palatable and provides a simple method of cleansing the gastrointestinal tract for patients. In particular, for a patient preparing to undergo a colonoscopy, the present composition and methods make the bowel preparation liquid taste significantly less salty.

The present invention provides a pharmaceutical composition for cleansing the gastrointestinal tract of a subject comprising one or more excipients and having a pH of the composition being adjusted in the range of from about 3.0 to about 4.0.

The invention further provides a method for cleansing the gastrointestinal tract of a subject. The method has the following steps: (1) determining the pH of a pharmaceutical composition for cleansing the gastrointestinal tract which has a salty taste and comprising one or more excipients, (2) adjusting the pH of the composition in the range from about 3.0 to about 4.0, and (3) orally administering the composition to the subject.

The present invention further provides a method of reducing the salty taste of a pharmaceutical composition for cleansing the gastrointestinal tract comprising adjusting the pH of the composition in the range of from about 3.0 to about 4.0, wherein the salty taste of the composition is reduced by at least about 30%, at least about 50%, or at least about 70%, compared to the salty taste of the composition had the pH not been adjusted in the range from about 3.0 to about 4.0.

The desired pH range is from about 3.0 to about 4.0, from about 3.1 to about 3.9, from about 3.2 to about 3.8, from about 3.3 to about 3.7, from about 3.4 to about 3.6, from or about 3.5.

The composition of the present invention is administered orally, and may be used to prepare of a procedure any part(s) of the gastrointestinal tract, including, but not limited to, the oesophagus, stomach, intestine (or bowel) such as the small intestine and the large intestine including cecum, colon and rectum. For example, the present methods may be used to empty the bowel.

The method of the present invention may be used to cleanse the gastrointestinal tract prior to a diagnostic, therapeutic and/or surgical procedure. Non-limiting examples of the surgical procedures include a colon surgery and a gastrointestinal tract surgery. Non-limiting examples of the diagnostic procedures include a barium enema examination, a capsule endoscopy, an endoscopy such as a colonoscopy or sigmoidoscopy. The colonoscopy can be conventional colonoscopy or virtual colonoscopy. (Heiken et al., Virtual colonoscopy for colorectal cancer screening: current status, November 2005, Cancer Imaging (International Cancer Imaging Society), 5 (Spec No A): S133-S139). The method of the present invention may be used in the treatment of acute gastrointestinal infections, for example bacterial or viral gastroenteritis. Colon cleansing is also useful for preventing infection after surgery on the lower intestine.

Perception of one or more tastes by a subject (such as a patient) may be assessed using suitable questionnaires or by personal interviews. For example, immediately after a subject consumes (or tastes) a composition (or other liquid compositions), he/she is asked to finish a questionnaire. In the questionnaire, the subject is required to judge the perceived salty taste (or other undesirable taste) intensity of the composition using, for example, a scale of 0 to 10 (or a scale of 0 to 100, a scale of 0 to 5, a scale of 0 to 9, etc.). The subject will be instructed that “0” represents “no intensity” or “minimal intensity” (i.e., no or minimal salty taste or other undesirable taste) whereas “10” (or “100,” etc.) represents the “highest intensity” of the salty taste (or other undesirable taste).

As taste is inherently subjective, the questionnaire or interview described above gives taste ratings that can be compared on the same patient or subject. The reduction in the undesirable taste (e.g., the salty taste) may be assessed by comparing the patient's (or subject's) taste rating when a taste-modifying substance was provided with the one when a taste-modifying substance was not provided. For example, if the taste rating without a taste-modifying substance is T1 and the taste rating with a taste-modifying substance is T2, the percentage reduction in the undesirable taste may be calculated as follows:

(T1−T2)/T1*100%

The undesirable taste of a liquid composition may be reduced by at least about 30%, at least about 40%, at least about 50%, at least about 60%, at least about 70%, at least about 80%, at least about 90%, compared to the undesirable taste of the liquid composition had the pH of the liquid not been adjusted in the range of about 3.0 to about 4.0.

One or more tastes can also be rated using the Thurstonian scale. A Thurstonian model is a latent variable model for describing the mapping of some continuous scale onto discrete, possibly ordered categories of response. In the model, each of these categories of response corresponds to a latent variable whose value is drawn from a normal distribution, independently of the other response variables and with constant variance. (Lawless et al., (1984), Direct and indirect scaling of sensory differences in simple taste and odor mixtures, J. Food Sci., 49, 44-51. Durlach, et al., (1969) Intensity Perception. I. Preliminary Theory of Intensity Resolution, Journal of the Acoustical Society of America, 46 (2): 372-383. Dessirier et al., 1998, Comparison of d′ values for the 2-AFC (paired comparison) and 3-AFC discrimination methods: Thurstonian models, sequential sensitivity analysis and power, Food Quality and Preference, 10 (1): 51-58. Frijter, J. E. R., (1980) Three-stimulus procedures in olfactory psychophysics: an experimental comparison of Thurstone-Ura and three-alternative forced choice models of signal detection theory, Perception & Psychophysics, 28 (5): 390-7. Gridgement, N. T., (1970) A Reexamination of the Two-Stage Triangle Test for the Perception of Sensory Differences, Journal of Food Science, 35 (1). Frijters, J. E. R., (1979) The paradox of discriminatory nondiscriminators resolved, Chemical Senses & Flavor 4 (4): 355-8. Valentin et al., Taste-odour interactions in sweet taste perception, In: Spillane W J, editor, Optimising sweet taste in foods. Cambridge (UK): Woodhead Publishing; 2006: 66-84).

Other rating scales can also be used in the present invention, including, but not limited to, intensity scales, just-about-right (JAR) scales and hedonic scale. In intensity scales, intensity rating questions ask respondents to rate the strength of a sensory attribute, for example, its saltiness, on a scale from “low” to “high”. In just-about-right scales, just-about-right questions ask respondents to rate whether the level of a sensory attribute, for example, its saltiness, is “too high”, “just right” or “too low”. Popper et al., The effect of attribute questions on overall liking ratings, Food Quality and Preference, 15 (2004) 853-858. Peryam et al., Advanced taste-test method, Food Eng., 1952, 24:58-61. Lim et al., Derivation and Evaluation of a Labeled Hedonic Scale, Chem. Senses, 34: 739-751, 2009. Gregson, R. A. M., A Rating-Scale Method for Determining Absolute Taste Thresholds, Journal of Food Science, 1962, 27: 376-380.

An exemplary intensity scale and an exemplary five-point JAR scale are shown in Table 1.

TABLE 1

| Intensity (Nine Points) | JAR (Five Points) |
|---|---|
| Extremely weak | Much too weak |
| | A little too weak |
| | Just about right |
| | A little too strong |
| | Much too strong |
| Extremely strong | |

The intensity of saltiness, sweetness, palatability or other tastes can be rated on a line scale, e.g., an unstructured or structured line scale, or other suitable line scales. In one embodiment, the rating scale is a 10-cm (or any other suitable length) visual analog scale with the anchors being “none” and “extremely strong”. All ratings reported in the visual analog scale are then converted to a 100-point scale by measuring the length of the segment marked by the test subject [Stevenson et al., Confusing tastes and smells: how odours can influence the perception of sweet and sour taste? Chem. Senses, 1999, 24: 624-635].

As used herein, the term “taste” refers to any taste including the five basic tastes (i.e., sweet, sour, salty, bitter and umami) and other tastes such as tart, alkaline, astringent, tangy, dry, sharp, cool, warm, hot, burning, acidic, spicy, pungent, kokumi, savory, tingling and/or metallic. Such taste shall include any and all taste(s) as well as any and all aftertaste(s). The list above is not all inclusive as one skilled in the art would recognize.

As used herein, the term “taste-modifying substance” or “taste-modifying agent” refers to any substance that is able to modify the perception of at least one taste during consumption of a composition (or a liquid composition). They may act to modify the perception of a taste or may affect the taste profile. The term “modify” means to change, alter, modulate, diminish, lessen, reduce, subdue, limit, intensify, supplement or potentiate. For example, a sour taste-modifying agent may modify the perception of a sour taste; a salty-modifying agent may modify the perception of a salty taste. A taste-modifying substance may or may not possess a taste(s) of its own.

In some embodiments, an increased sensation of another taste may allow the blocking of an undesirable taste. For example, an increased sweet sensation that is perceived by the addition of a taste-modifying substance may diminish a salty taste.

A taste-modifying substance may function by modulating the activity of taste receptor cells and/or the taste signalling pathway in a mammal. Specifically, taste is perceived through sensory cells located in the taste buds. Different signalling mechanisms sense the primary tastes of salty, sour, sweet, bitter and umami. Eventually a nerve impulse is triggered in the brain that is sensed as one of these primary tastes. For instance, in some cases, taste-modifying substances may bind to taste receptors, such as sweet taste receptors, which thereby modify the perception of the sweet taste. In other embodiments, taste-modifying substances may block taste receptors, such as salty receptors, which suppress the perception of a salty taste.

The effect of a taste-modifying substance to modify the perception of a taste may or may not depend on its concentration. A taste-modifying substance may be used alone or in combination with other taste-modifying substance(s). When two or more taste-modifying substances are used, they may act additively or synergistically.

There may exist differences in taste perception between individuals. For example, there can be more than one perception of a single taste, whether such taste is a basic taste or another taste. For example, there may be a number of different “salty” tastes that can be noted by some individuals. U.S. Pat. No. 6,015,792.

The taste-modifying substance that may be used in the composition of the present invention can be a sweet taste-modifying agent, a sour taste-modifying agent, a salty taste-modifying agent, a bitter taste-modifying agent, an umami taste-modifying agent, etc.

Non-limiting examples of the taste-modifying substance include thaumatin, monellin, mabinlin, brazzein, pentadin, curculin, neuculin, miraculin and mixtures thereof. The taste-modifying substance can be provided in the form of a capsule, a tablet, a pill, granules, powders, a pellet, a solids mixture, a solution, a dispersion, an emulsion, a paste, an extract, or an isolate from a natural source. Preferably, the taste-modifying substance is present in the composition.

The taste-modifying substance may be present in amounts ranging from about 0.01% to about 100%, from about 0.1% to about 90%, from about 1% to about 80%, from about 5% to about 70%, from about 5% to about 60%, from about 5% to about 50%, from about 5% to about 40%, or from about 5% to about 30% by weight of the composition containing the taste-modifying substance.

The composition of the present invention is administered orally, and may be used to prepare any part(s) of the gastrointestinal tract, including, but not limited to, the esophagus, stomach, intestine (or bowel) such as the small intestine and the large intestine including cecum, colon and rectum.

Bowel preparation compositions, also called bowel cleansers, bowel cleansing compositions purgatives, cathartics, and lavages, are formulated for rapid emptying of the bowel and are intended for short-term use. Bowel preparation compositions include, for example, colon evacuants and colon cleansing compositions.

The composition of the present invention may be isosmotic or hyperosmotic. The composition may contain one or more electrolytes. The composition may contain at least one salt, including, but not limited to, a sodium salt, a potassium salt, a calcium salt, an ammonium salt or mixtures thereof. For example, the salts may be sodium chloride, potassium chloride, sodium bicarbonate (sodium hydrogen carbonate), sodium sulfate, sodium phosphate or mixtures thereof. The preparation composition may comprise at least one alkali metal salt, and/or at least one alkaline earth metal salt. The alkali metal may be sodium, potassium, etc. The alkaline earth metal may be magnesium, calcium, etc.

The composition may contain at least one sodium phosphate. The composition may have sodium phosphate in varying proportions of monobasic and dibasic species.

The concentration of the salt in the composition of the invention may vary depending on the type of the salt and other factors. For example, a litre of the preparation composition may contain greater than about 0.2 g, greater than about 0.5 g, greater than about 1 g, greater than about 2 g, greater than about 3 g, greater than about 5 g, less than about 10 g, less than about 9 g, less than about 7.5 g, less than about 7 g, less than about 5 g, less than about 4 g, less than about 2 g, or less than about 1.5 g of a salt.

The composition may comprise polyethylene glycol (PEG). The PEG may comprise any food-grade or pharmaceutical-grade PEG. The average molecular weight of PEG may be greater than about 900, greater than about 2000, greater than about 2500, less than about 4500, or between about 3000 and about 8000. For example, it may be PEG 4000 or PEG 3350. PEG may also be lower molecular weight PEG polymers (such as PEG 400). The PEG used in a composition of the invention may comprise one PEG species, or two or more different PEG species.

The concentration of PEG in the composition of the invention may vary. A litre of the composition of the invention may contain greater than about 20 g, greater than about 800 g, less than about 250 g, less than about 150 g, less than about 140 g, or less than about 80 g of PEG. For example, a composition of the invention may comprise less than about 80 g per litre of PEG.

The composition may be administered over a time period ranging from about 30 minutes to about 3 days, from about 1 hour to about 24 hours, from about 2 hours to about 12 hours, or from about 1 hour to about 4 hours. The administration time period may be in a continuous period or a discontinuous period. In discontinuous administrations, a portion of the composition, for example, approximately half of the composition, may be administered the evening before the diagnostic, therapeutic or surgical procedure is to be carried out, with the remainder of the composition being administered on the day of the procedure. The composition may be taken once or several times per day on the day of the diagnostic, surgical or therapeutic procedure, and/or on the day(s) preceding the procedure, depending upon various factors, such as the procedure, the degree of cleansing required, and the patient's condition (e.g., the presence of complicating bowel conditions such as constipation).

The composition may be a liquid or a solid.

When the composition is a liquid (e.g., a solution), the dose or volume of the composition to be administered will depend on the patient being treated. For example, a smaller dose or volume of preparation solution is appropriate in the treatment of small children and a higher volume of preparation solution is appropriate in adult patients. When the composition is a liquid, the volume of the composition administered may range from about 0.1 litres to about 5 litres, from about 0.2 litres to about 4.5 litres, from about 0.5 litres to about 4 litres, from about 1 litre to about 4 litres, from about 1 litre to about 3 litres, or from about 1.5 litres to about 2 litres. A patient may be required to finish all the composition, or may be asked to take the preparation composition until, for example, the rectal effluent is clear.

When the composition is a solution, it may have any suitable osmolarity, for example, greater than about 200 mOsmol/kg, greater than about 250 mOsmol/kg, from about 250 mOsmol/kg to about 350 mOsmol/kg, from about 200 mOsmol/kg to about 800 mOsmol/kg, from about 300 mOsmol/kg to about 700 mOsmol/kg, from about 330 mOsmol/kg to about 600 mOsmol/kg, from about 350 mOsmol/kg to about 600 mOsmol/kg, from about 460 mOsmol/kg to about 550 mOsmol/kg, from about 350 mOsmol/kg to about 470 mOsmol/kg, from about 250 mOsmol/kg to about 470 mOsmol/kg, from about 250 mOsmol/kg to about 500 mOsmol/kg, from about 250 mOsmol/kg to about 550 mOsmol/kg, greater than about 330 mOsmol/kg, greater than about 350 mOsmol/kg, greater than about 400 mOsmol/kg, greater than about 460 mOsmol/kg, less than about 600 mOsmol/kg, less than about 550 mOsmol/kg, less than about 500 mOsmol/kg, and less than about 470 mOsmol/kg.

The composition may also be concentrate compositions, such as, in dry form (e.g., powder, tablet, granular or any other suitable physical form) or in liquid form (e.g., syrup, suspension or emulsion). The bulk of the liquid component of a finished composition is not present in the concentrate to allow for reduced weight, volume, storage and shipping costs while at the same time allowing for increased shelf life of the concentrate versus final, diluted composition. When preparing the final, ready-to-administer composition, the concentrate composition may be diluted by any suitable liquid, such as water, tea, etc.

The methods of the invention may also include administering additional agents to the patient. For example, for added potency in certain clinical applications, a bowel stimulant such as biscodyl, or other agent known for its laxative properties may be taken in conjunction with the administration of these compositions as appropriate.

The composition may contain at least one pharmaceutical carrier according to conventional pharmaceutical techniques. The carrier may take a wide variety of forms depending on the form of preparation desired for administration. For example, for liquid oral preparations such as, suspensions, elixirs and solutions, suitable carriers and additives include water, glycols, oils, alcohols, flavoring agents, preservatives, coloring agents and the like. For solid oral preparations such as, powders, capsules and tablets, suitable carriers and additives include starches, sugars, diluents, granulating agents, lubricants, binders, disintegrating agents and the like. In some embodiments, the composition may include optional additives such as antioxidants, amino acids, caffeine, emulsifiers, minerals, micronutrients, phytochemicals (“phytonutrients”), stabilizers, thickening agents, medicaments, vitamins, or mixtures thereof.

The pH of a solution (or a liquid composition) may be measured using conventional laboratory techniques, such as using a pH meter, pH sensor, pH indicator, pH test paper etc. In certain embodiments, it is also possible to calculate pH from knowledge of the components of a solution.

The solution may be acidified by at least an inorganic acid or an organic acid including, but not limited to, citric acid, acetic acid, phosphoric acid, malic acid, succinic acid, formic acid, fumaric acid, maleic acid, adipic acid, butyric acid, glyconic acid, lactic acid, oxalic acid, tartaric acid and mixtures thereof, or other permitted food acids.

The pH of the solution may also be adjusted by compounds including, but not limited to, ammonium hydroxide, sodium carbonate, potassium carbonate, sodium bicarbonate, carbon dioxide, and mixtures thereof.

When adjusting the pH of the composition, the pH may be increased or decreased by at least about 0.05 pH units, at least about 0.1 pH units, at least about 0.15 pH units, at least about 0.2 pH units, at least about 0.3 pH units, at least about 0.4 pH units, or at least about 0.5 pH units.

### EXAMPLE 1

### Sodium Picosulfate, Magnesium Oxide, Anhydrous Citric Acid Powder for Oral Solution

TABLE 1

| Sr. No. | Ingredient | Quantity (mg/Pouch) |
|---|---|---|
| 1 | Sodium Picosulfate | 10 |
| 2 | Magnesium Oxide | 3500 |
| 3 | Citric Acid Anhydrous | 50,000-100,000 |
| 4 | Potassium Bicarbonate | 500 |
| 5 | Sodium Saccharin | 50 |
| 6 | Orange Flavour | 40 |
| | Total | 16,100 |

Process: Magnesium Oxide and Citric Acid Anhydrous were screened, mixed and blended. The mixture was subjected to roller compaction followed by milling to form granules.

Half of the quantity of the milled granules were blended with Sodium Picosulfate. The remaining quantity of the milled granules was blended with Potassium Bicarbonate, Sodium Saccharin, and Orange Flavour. The final mixture was then filled in pouches.

The pH of the mixture measured upon reconstituting in water was in the range of 3.0 to 4.0.

### Ingredient Specifications

(A) Input Sodium Picosulfate PSD: 100% of the particles less than 30 μm.

(B) Input Citric Acid PSD: 250 μm to 600 μm.

(C) Final Blend PSD: 50% of final blend particles are less than 75 μm and 50% of final blend particles are between 100 μm to 400 μm.

### EXAMPLE 2

### Sodium Picosulfate, Magnesium Oxide, Anhydrous Citric Acid Powder for Oral Solution

The formulation summarized in Table 1 of Example 1 was prepared by the following process—

Sodium Picosulfate, Magnesium Oxide and Citric Acid Anhydrous were screened, mixed and blended. The mixture was subjected to roller compaction followed by milling to form granules.

The milled granules were blended with Potassium Bicarbonate, Sodium Saccharin, and Orange Flavour. The final mixture was then filled in pouches.

The pH of the mixture measured upon reconstituting in water was in the range of 3.0 to 4.0.

### EXAMPLE 3

### Sodium Picosulfate, Magnesium Oxide, Anhydrous Citric Acid Powder for Oral Solution

The formulation summarized in Table 1 of Example 1 was prepared by the following process—

Magnesium Oxide and Citric Acid Anhydrous were screened, mixed and blended. Sodium Picosulfate was then added and blended with the mixture. The mixture was subjected to roller compaction followed by milling to form granules.

The milled granules were blended with Potassium Bicarbonate, Sodium Saccharin, and Orange Flavour. The final mixture was then filled in pouches.

The pH of the mixture measured upon reconstituting in water was in the range of 3.0 to 4.0.

### EXAMPLE 4

### Sodium Picosulfate, Magnesium Oxide, Anhydrous Citric Acid Powder for Oral Solution

The formulation summarized in Table 1 of Example 1 was prepared by the following process—

Magnesium Oxide and a large amount of the total Citric Acid Anhydrous were screened, mixed and blended. The mixture was subjected to roller compaction followed by milling to form granules.

Separately, Sodium picosulfate and the remaining amount of the Citric Acid Anhydrous were screened, mixed and blended. The mixture was then milled and mixed with milled granules. To the blend was further added Potassium Bicarbonate, Sodium Saccharin and Orange Flavour. The final mixture was then filled in pouches.

The pH of the mixture measured upon reconstituting in water was in the range of 3.0 to 4.0.

### EXAMPLE 5

### Sodium Picosulfate, Magnesium Oxide, Anhydrous Citric Acid Powder for Oral Solution

The formulation summarized in Table 1 of Example 1 was prepared by the following process—

Magnesium Oxide and a large amount of the total Citric Acid Anhydrous were screened, mixed and blended. The mixture was subjected to roller compaction followed by milling to form granules.

Separately, the remaining amount of the Citric Acid Anhydrous was screened and milled followed by addition of Sodium picosulfate. Potassium Bicarbonate, Sodium Saccharin and Orange Flavour were then added to the mixture. Finally milled granules were added to the said mixture. The mixture was then filled in pouches.

The pH of the mixture measured upon reconstituting in water was in the range of 3.0 to 4.0.

### EXAMPLE 6

### Sodium Picosulfate, Magnesium Oxide, Anhydrous Citric Acid Powder for Oral Solution

The formulation summarized in Table 1 of Example 1 was prepared by the following process—

Magnesium Oxide and a large amount of the total Citric Acid Anhydrous were screened, mixed and blended. The mixture was subjected to roller compaction followed by milling to form granules.

Separately, the remaining amount of the Citric Acid Anhydrous, Sodium picosulfate and Sodium Saccharin were screened, mixed and milled. Potassium Bicarbonate, Sodium Saccharin and Orange Flavour were then added to the mixture. Finally milled granules were added to the mixture and the mixture was then filled in pouches.

The pH of the mixture measured upon reconstituting in water was in the range of 3.0 to 4.0.

### EXAMPLE 7

### Sodium Picosulfate, Magnesium Oxide, Anhydrous Citric Acid Powder for Oral Solution

The formulation summarized in Table 1 of Example 1 was prepared by the following process—

(1) Magnesium Oxide and a large amount of the Citric Acid Anhydrous were screened, mixed and blended. The mixture was subjected to roller compaction followed by milling to form granules.

(2) Separately, the remaining amount of the Citric Acid Anhydrous was screened and milled followed by addition of Sodium picosulfate. The mixture was subjected to roller compaction followed by milling to form granules. Potassium Bicarbonate, Sodium Saccharin and Orange Flavour were then added to the milled granules mixture.

(3) Finally milled granules prepared in step (1) and blend prepared in step (2) were mixed and the mixture was then filled in pouches.

The pH of the mixture measured upon reconstituting in water was in the range of 3.0 to 4.0.

What is claimed is:

1. A pharmaceutical composition for cleansing the gastrointestinal tract of a subject, the composition having the pH adjusted in the range of from about 3.0 to about 4.0 and the composition is at least about 30% more palatable than the composition in which pH is not adjusted in the range of from about 3.0 to about 4.0, wherein said composition is devoid of ascorbic acid, a salt of ascorbic acid, or a mixture thereof.

2. The composition of claim 1, wherein said composition is in the form of a bowel preparation solution.

3. The composition of claim 2, wherein the volume of the said solution is less than 1 litre.

4. The composition of claim 2, wherein said solution is prepared by reconstituting said composition in water.

5. The composition of claim 4, wherein said solution exhibits a pH in the range of from about 3.0 to about 4.0.

6. The composition of claim 1, wherein said composition further comprises one or more inorganic acids, organic acids or mixtures thereof.

7. The composition of claim 6, wherein said inorganic acid and/or organic acid in the composition are selected from the group consisting of citric acid, acetic acid, phosphoric acid, malic acid, succinic acid, formic acid, fumaric acid, maleic acid, adipic acid, butyric acid, glyconic acid, lactic acid, oxalic acid, tartaric acid and mixtures thereof.

8. The composition of claim 1, wherein said composition is devoid of a taste-modifying substance.

9. The composition of claim 1, wherein said composition further comprises one or more taste-modifying substances.

10. The composition of claim 1, wherein said composition further comprises polyethylene glycol in amount from about 20 gm to about 350 gm per litre of the composition.

11. The composition of claim 10, wherein the polyethylene glycol in said composition is present in amount of less than 80 gm per litre of the composition.

12. The composition of claim 1, wherein said composition further comprises one or more alkali or alkaline earth metal sulfate salts.

13. The composition of claim 1, wherein said composition further comprises one or more sugars.

14. The composition of claim 13, wherein sugar in said composition is present in an amount of less than 1.5 gm per litre of the composition.

15. A method of cleansing the gastrointestinal tract prior to an endoscopy procedure, wherein the method comprises of orally administering the composition of claim 1.

16. The method of claim 15, wherein the endoscopy procedure comprises colonoscopy, sigmoidoscopy, a barium enema examination, capsule endoscopy, colon surgery or gastrointestinal tract surgery

17. A method for cleansing the gastrointestinal tract of a subject comprising the steps of:
(a) determining the pH of a pharmaceutical composition for cleansing the gastrointestinal tract, wherein the composition has a salty taste and includes one or more excipients;
(b) adjusting pH of the composition to be in the range of from about 3.0 to about 4.0; and
(c) orally administering the composition to the subject, wherein,
(i) said composition is devoid of ascorbic acid, a salt of ascorbic acid, or a mixture thereof; and
(ii) the salty taste of the composition is reduced by at least about 30% compared to the salty taste of the composition had the pH not been adjusted to be in the range of from about 3.0 to about 4.0.

18. The method of claim 17, wherein the salty taste of the composition is reduced by at least about 50%.

19. A pharmaceutical composition for cleansing the gastrointestinal tract of a subject consisting essentially of:
(a) polyethylene glycol,
(b) an alkali or alkaline earth metal sulfate,
(c) at least one inorganic acid and/or organic acid; and
(d) optionally one or more electrolytes selected from sodium chloride, potassium chloride, sodium hydrogen carbonate, potassium hydrogen carbonate, and magnesium oxide;
wherein,
(i) said organic acid is not ascorbic acid, a salt of ascorbic acid, or a mixture thereof, and
(ii) the pH of said composition is in the range of from about 3.0 to about 4.0.

20. The composition of claim 10, wherein the salty taste of the composition is reduced by at least about 30% to about 50%.
